(19) United States
(12) Patent Application Publication
(10) Pub. No.: US 2007/0203190 A1
(43) Pub. Date: Aug. 30, 2007

(54) Hydroxylamines and derivatives for the inhibition of complement activation

(76) Inventors: Ghanshyam Patil, Lincoln University, PA (US); William L. Matier, Hockessin, DE (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/677,462

(22) Filed: Feb. 21, 2007

Related U.S. Application Data
(60) Provisional application No. 60/775,456, filed on Feb. 22, 2006.
(60) Provisional application No. 60/775,478, filed on Feb. 22, 2006.

(51) Int. Cl. A61K 31/445; A61K 31/421

(52) U.S. Cl. 514/327; 514/376

(57) ABSTRACT

Methods for the inhibition of complement activation, for the treatment of complement-mediated pathologies, and for the treatment of drusen-mediated pathologies are disclosed. The methods utilize hydroxylamine compounds and ester derivatives thereof, administered to subjects in effective amounts.

### CROSS REFERENCE TO RELATED APPLICATION

This application claims priority to U.S. Provisional Application Ser. No. 60/775,478, filed Feb. 22, 2006, the disclosure of which is incorporated herein by reference in its entirety.

### FIELD OF THE INVENTION

The invention relates generally to the field of immunopharmacology. Specifically, the invention features compositions and methods utilizing hydroxylamines and certain ester derivatives for inhibiting the activation of the complement cascade and for treating pathologies that result from complement activation.

### BACKGROUND OF THE INVENTION

Various patents and other publications are referenced herein. The contents of each of these patents and publications are incorporated by reference herein, in their entireties. The entire contents of commonly-owned co-pending U.S. Publication Nos. 2004/0002461, 2005/0130906 and 2005/0131025 are incorporated by reference herein.

The complement system is an important weapon in the body's arsenal for immunological defense against foreign pathogens. Complement proteins are activated in an enzyme cascade that can be triggered by various signals, and proceed through one of three main pathways, termed the classical, alternative or lectin pathways. These pathways result in the generation of anaphylatoxic peptides, including C3a and C5a, and can culminate in the formation of the C5b-9 membrane attack complex (MAC), which functions to lyse invading cells. The anaphylatoxins can exert their effects on blood vessels, facilitating inflammation as well as the contraction of smooth muscle and an increase in vascular permeability.

In certain situations, the complement system can produce deleterious effects. For example, inappropriate activation of complement may result in damage to endogenous cells. Complement can exacerbate damage to tissues in antibody-mediated autoimmune diseases such as myasthenia gravis and systemic lupus erythematosus, especially when immune complexes are produced, and can exacerbate tissue damage following ischemia (Liszewski M K et al. (1998) Expert Opin. Investig. Drugs. 7:323-31). Complement has also been implicated in facilitating or exacerbating various disease states, including glomerulonephritis, adult respiratory syndrome, and rejection of transplantated tissues (Glovsky M M et al. (2004) Ann. Allergy Asthma Immunol. 93:513-22; Colvin R B et al. (2005) Nat Rev Immunol. 5:807-17). Complement-mediated tissue injury has also been found to result from bioincompatibility situations, such as those encountered in patients undergoing dialysis or cardiopulmonary bypass (Mollnes T E (1998) Vox Sang. 74 Suppl 2:303-307).

Complement-mediated tissue injuries are directly mediated by the MAC, and indirectly by the generation of the anaphylatoxins C3a and C5a. These peptides induce damage through their effects on neutrophils and mast cells. Regulation of complement at the C3 and C5 activation steps is provided by both plasma and membrane proteins. The plasma protein inhibitors include factor H and C4-binding protein, and the regulatory membrane proteins located on cell surfaces include complement receptors 1 (CR1), decay-accelerating factor (DAF), and membrane cofactor protein (MCP). These proteins inhibit the C3 and C5 convertases (multi-subunit proteases), by promoting dissociation of the multisubunit complexes and/or by inactivating the complexes through proteolysis (catalyzed by factor I).

Complement has also been implicated in drusen formation. Drusen is the name given to extracellular deposits localized to the area of the eye between the retinal pigmented epithelium (RPE) and Bruch's membrane, and sometimes localized to the retinal periphery (Lewis H B et al. (1986) Ophthalmology 93:1098-1111). Drusen contains various lipids, proteins, polysaccharides, and glycosaminoglycans, and drusen proteins are often found oxidatively modified (Crabb J W et al. (2002) Proc. Natl. Acad. Sci. USA 99: 14682-7). Drusen deposition occurs primarily in aged individuals, and is a primary factor in the pathogenesis of age related macular degeneration (AMD) (Abdelsalam A et al. (1999) Surv. Opthalmol. 44:1-29).

Although the precise mechanisms that lead to drusen formation and depositions have only been partially characterized, there has been speculation that cellular debris from the RPE serves as a stimulus for inflammation and in turn provides a potential nucleation site for the accumulation of drusen (Johnson L V et al. (2000) Exp. Eye Res. 70:441-9; and, Johnson L V et al. (2001) Exp. Eye. Res. 73:887-96). In support of this hypothesis, various inflammatory mediators, including the complement constituents C3a, C5a, and the MAC have been observed in drusen (Luibl V et al. (2006) J. Clin. Invest. 116:378-85), and such components have been found to be colocalized with a complement-activating protein, amyloid-beta protein, in substructural vesicles within drusen (Johnson L V et al. (2002) Proc. Natl. Acad. Sci. USA 99:11830-5). In addition, recent work has demonstrated neutralization of C3a or C5a or their respective receptors reduced neovascularization in AMD (Nozaki M et al. (2006) Proc. Natl. Acad. Sci. USA 2006 Feb. 1; [electronic publication ahead of print]. These observations indicate that complement may play a role in the initiation or progression of drusen formation and deposition. As such, complement is an attractive target for inhibiting drusen deposition.

To date, there are no clinically viable inhibitors of complement activation although certain candidates for clinical use exist. Such candidates include a recombinant form of complement receptor 1 known as soluble complement receptor 1 (sCR1) and a humanized monoclonal anti-C5 antibody (5G1.1-scFv). Both of these substances have been shown to suppress complement activation in in vivo animal models (Kalli KR et al. (1994) Springer Semin. Immunopathol. 15:417-31; and, Wang et al. (1996) Proc. Natl. Acad. Sci. U S A. 93:8563-8). However, each substance possesses the disadvantage of being large molecular weight proteins (240 kDa and 26,000 kDa, respectively) that are difficult to manufacture and must be administered by infusion. CD59, which blocks assembly of the MAC, has also been proposed as a potential therapeutic agent, but has shown limited activity in vitro (Song H et al. (2003) J. Clin. Invest. 111:1875-85). Accordingly, recent research has emphasized the development of smaller active agents that are easier to deliver, more stable, and less toxic to the patient to which they are administered.

Due to their comparative lack of toxicity, hydroxylamines are preferable to nitroxides as therapeutic agents. Published United States Patent Applications 2004/0002461, 2005/0130906 and 2005/0131025 to Matier and Patil disclose hydroxylamines and related compounds and their use in the treatment of a variety of ophthalmic conditions in which oxidative damage or inflammation are involved. Such compounds possess numerous advantageous qualities, including robust anti-inflammatory and antioxidant activities, as well as ocular permeability in some instances. However, hydroxylamines heretofore have not been reported as possessing any efficacy to inhibit complement activation, or to treat complement- or drusen-mediated pathologies such as AMD.

### SUMMARY OF THE INVENTION

The present invention features methods for inhibiting complement activation in a subject by administering to the subject a hydroxylamine compound or ester derivative thereof in an amount effective to inhibit complement activation. In addition, the invention provides methods for treating pathologies mediated by complement activation in a subject and methods for inhibiting drusen formation in a subject by administering to the subject a composition comprising a pharmaceutically acceptable carrier and a hydroxylamine compound or ester derivative thereof in an amount effective to treat pathologies mediated by complement activation and to inhibit drusen formation, respectively. Examples of ophthalmic pathologies that can be treated by the compositions and methods of the invention include retinopathy and age-related macular degeneration (AMD). Moreover, it is becoming apparent that chronic inflammation, such as results from complement activation, is factor in many of the important diseases of aging. Accordingly, other pathologies that can be treated by the compositions and methods of the invention include age-related disorders, such as Alzheimer's disease (AD), Parkinson's disease (PD), ALS and multiple sclerosis, atherosclerosis, heart disease, skin elastosis, glomerular basement membrane disease and numerous amyloidoses, to name a few. The compositions and methods can be used in any animal, and preferably are used in mammals, and most preferably are used in humans.

In certain embodiments, the hydroxylamine compounds include Tempol-H, Tempo-H, and Oxano-H, and the derivatives of the hydroxylamine compounds have formula I: 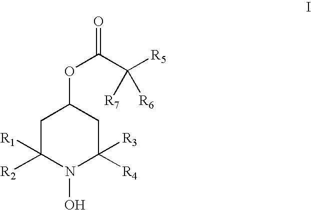
wherein:

R₁ and R₂ are, independently, H or C₁ to C₃alkyl;

R₃ and R₄ are, independently C₁ to C₃alkyl, or wherein R₁ and R₂, taken together, or R₃ and R₄, taken together, or R₁ and R₂, taken together and R₃ and R₄ taken together, are each cycloalkyl;

R₅ is H, OH, or C₁ to C₆alkyl;

R₆ is or C₁ to C₆alkyl, alkenyl, alkynyl, or substituted alkyl or alkenyl;
R₇ is C₁ to C₆alkyl, alkenyl, alkynyl, or substituted alkyl or alkenyl; or R₆ and R₇ taken together, or R₅, R₆, and R₇ taken together, form a carbocycle having from 3 to 7 atoms in the ring or form a heterocycle having from 3 to 7 atoms in the ring.

In some aspects of the invention, the compositions of the invention are used synergistically with other complement inhibitors, anti-inflammatory agents, or with antioxidants.

Other features and advantages of the invention will be understood by reference to the drawings, detailed description, and examples that follow.

### DETAILED DESCRIPTION OF ILLUSTRATIVE EMBODIMENTS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The following abbreviations may be used in the specification and examples: AMD, age-related macular degeneration; MAC, membrane attack complex.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters; including the results of a physical examination, neurological examination, and/or psychiatric evaluations.

“Effective amount” or “therapeutically effective amount” are used interchangeably herein, and refer to an amount of a compound, material, or composition, as described herein effective to achieve a particular biological result. Such results may include, but are not limited to, the inhibition of complement activation, the inhibition of drusen formation, the treatment of complement-mediated pathologies, and the treatment of drusen-mediated pathologies.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

“Pathology” refers to the structural and functional deviations from a normal state that constitute the inception or progression of a disorder, disease, or disease state, or characterize a particular disorder or disease.

“Drusen” refers to any extracellular deposits that accumulate beneath the basement membrane of the retinal pigmented epithelium (RPE) and the inner collagenous layer of the Bruch membrane.

The present invention provides methods for the inhibition of complement activation. Also provided are methods for the treatment of pathologies mediated by complement activation and pathologies mediated by drusen formation and deposition. The methods comprise administration a hydroxylamine compound or ester derivative thereof in an amount effective to treat, inhibit, or slow the activation of complement, the deposition of drusen, or the onset or progression of complement- or drusen-mediated pathologies.

The invention further provides hydroxylamine compounds, including Tempol-H, Tempo-H, and Oxano-H, and any pharmaceutically acceptable salts, analogs, homologs, conjugates, and derivatives thereof in the manufacture of a medicament for the treatment or inhibition of pathologies mediated by complement activation, or pathologies mediated by the formation and deposition of drusen, including age-related macular degeneration and diabetic retinopathy.

Preferred examples of the type of hydroxylamine compounds suitable for use in the present invention are TEMPOL-H (TPH, the hydroxylamine reduced form of the nitroxide 4-hydroxy-2,2,6,6-tetramethylpiperidin-1-yloxy), TEMPO-H (the hydroxylamine reduced form of the nitroxide 2,2,6,6-tetramethylpiperidin-1-yloxy) and OXANO-H (2-Ethyl-2,4,4-trimethyl-oxazolidin-3-ol), which is the reduced form of OXANO, 2-ethyl-2,4,4-trimethyloxazolidin-3-yloxy). Other hydroxylamine compounds suitable for use in the present invention include, but are not limited to, those disclosed by Hahn et al (1998, supra; 2000, supra), Samuni et al (2001, supra); and in U.S. Pat. No. 5,981,548 to Paolini, et al. (disclosing certain N-hydroxylpiperidine esters and their use as antioxidants in a number of contexts); U.S. Pat. No. 4,404,302 to Gupta et al. (disclosing the use of certain N-hydroxylamines as light stabilizers in plastics formulations); U.S. Pat. No. 4,691,015, to Behrens et al. (describing hydroxylamines derived from hindered amines and the use of certain of them for the stabilization of polyolefins); and the hydroxylamine compounds disclosed in the several aforementioned U.S. patents to Hsia et al.; and the hydroxylamine counterparts of the nitroxides disclosed in U.S. Pat. Nos. 5,462,946 and 6,605,619 to Mitchell et al., namely, (1) compounds of the formula R₃—N(R₄)(R₅) wherein R₃ is —OH and R₄ and R₅ combine together with the nitrogen to form a heterocycle group, or wherein R₄ and R₅ themselves comprise a substituted or unsubstituted cyclic or heterocyclic group; (2) metal-independent hydroxylamines of formula R₃—N(R₄)(R₅) wherein R₃ is —OH and R₄ and R₅, together with the nitrogen atom to which they are bonded, form a 5- or 6-membered heterocyclic group, which, in addition to said nitrogen atom, comprises one or more heteroatoms selected from the group consisting of oxygen, nitrogen and sulfur, or R₄ and R₅, separately, each comprise a substituted or unsubstituted 5- or 6-membered cyclic group or a substituted or unsubstituted 5- or 6-membered heterocyclic group, which comprises one or more heteroatoms selected from the group consisting of oxygen, nitrogen and sulfur; or (3) oxazolidine compounds of the formula:

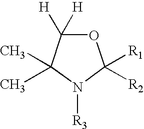

wherein R₁ is —CH₃ and R₂ is —C₂H₅, —C₃H₇, —C₄H₉, —C₅H₁₁, —C₆H₁₃, —CH₂CH(CH₃)₂, —CHCH₃C₂H₅, or —(CH₂)₇CH₃, and R₃ is —OH, or wherein R₁ and R₂ together form spirocyclopentane, spirocyclohexane, spirocycloheptane, spirocyclooctane, 5-cholestane or norbornane; and pharmaceutically acceptable salts of any of the above-listed compounds. Insofar as is known, the above-referenced compounds have not been used heretofore for treating or inhibiting hepatitis.

Ester derivatives of hydroxylamines suitable for use in the present invention comprise compounds of formula I or their pharmaceutically acceptable salts, examples of which are described in detail in U.S. Published Application 2004/0002461: 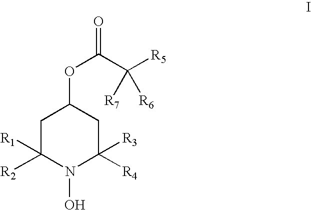
wherein:

R₁ and R₂ are, independently, H or C₁ to C₃alkyl;

R₃ and R₄ are, independently C₁ to C₃alkyl, or wherein R₁ and R₂, taken together, or R₃ and R₄, taken together, or R₁ and R₂, taken together and R₃ and R₄ taken together, are each cycloalkyl;

R₅ is H, OH, or C₁ to C₆alkyl;

R₆ is or C₁ to C₆alkyl, alkenyl, alkynyl, or substituted alkyl or alkenyl;

R₇ is C₁ to C₆alkyl, alkenyl, alkynyl, or substituted alkyl or alkenyl; or R₆ and R₇ taken together, or R₅, R₆, and R₇ taken together, form a carbocycle having from 3 to 7 atoms in the ring or form a heterocycle having from 3 to 7 atoms in the ring.

The methods of the present invention may also utilize compositions comprising a pharmaceutically acceptable carrier or diluent and a hydroxylamine compound having an N-hydroxy piperidine portion bound to a solubility modifying portion, the compound having a solubility in water at 25° C. of at least about 0.25% by weight and a water/n-octanol partition coefficient at 25° C. of at least about 5. The composition may have the N-hydroxy piperidine portion cleavable from the compound under conditions found in biological tissues, such as found in the eye. The N-hydroxy piperidine portion may be cleaved enzymatically. The compositions may also exist wherein the N-hydroxy piperidine portion is 1-oxyl-4-hydroxy-2,2,6,6-tetramethylpiperidyl.

The term C₁ to Cₙ alkyl, alkenyl, or alkynyl, in the sense of this invention, means a hydrocarbyl group having from 1 to n carbon atoms in it, wherein n is an integer from 1 to about 20, preferably 1 to about 10, yet more preferably, 1 to about 6, with from 1 to about 3 being even more preferred. The term thus comprehends methyl, ethyl, n-propyl, iso-propyl, n-butyl, sec-butyl, iso-butyl, tert-butyl, and the various isomeric forms of pentyl, hexyl, and the like. Likewise, the term includes ethenyl, ethynyl, propenyl, propynyl, and similar branched and unbranched unsaturated hydrocarbon groups of up to n carbon atoms. As the context may admit, such groups may be functionalized such as with one or more hydroxy, alkoxy, alkylthio, alkylamino, dialkylamino, aryloxy, arylamino, benzyloxy, benzylamino, heterocycle, or YCO-Z, where Y is O, N, or S and Z is alkyl, cycloalkyl, heterocycle, or aryl substituent.

The term carbocycle defines cyclic structures or rings, wherein all atoms forming the ring are carbon. Exemplary of these are cyclopropyl, cyclobutyl, cyclopentyl, cyclohexyl, cycloheptyl, etc. Cyclopropyl is one preferred species. Heterocycle defines a cyclic structure where at least one atom of the ring is not carbon. Examples of this broad class include furan, dihydrofuran, tetrahydrofuran, pyran, oxazole, oxazoline, oxazolidine, imidazole and others, especially those with an oxygen atom in the ring. Five, six and seven membered rings with at least one oxygen or nitrogen atom in the ring are preferred heterocycles. Furanyl and tetrahydrofuranyl species are among those preferred.

It is preferred for certain embodiments that each of R₁ through R₄ be lower alkyl that is C₁ to C₃ alkyl. Preferably, all these groups are methyl for convenience in synthesis and due to the known efficacy of moieties having such substitution at these positions. However, other substituents may be used as well.

In certain embodiments, compounds are employed where R₆ is C₁ to C₆ alkyl substituted with at least one C₁ to C₆ alkoxy or benzyloxy group. Preferred among these are compounds having ethoxy or benzyloxy substituents. Among preferred compounds are those where each of R₁ through R₄ is methyl, R₅ is H or methyl, R₆ is methyl substituted with benzyloxy or C₁ to C₆ alkoxy, and R₇ is methyl or where R₆ and R₇ form a cyclopropyl group as well as the compound in which each of R₁ through R₄ is methyl, R₅ is methyl, R₆ is ethoxy or benzyloxy methyl, and R₇ is methyl. An additional preferred compound is one in which each of R₁ through R₄ is methyl, R₅ is methyl, R₆ is hydroxymethyl, and R₇ is methyl.

Other useful compounds are those wherein each of R₁ through R₄ is methyl, and R₅, R₆, and R₇ form a furanyl group, or in which R₆ and R₇ form a tetrahydrofuranyl group. The compound where R₁ through R₄ is methyl, R₅ is H and, R₆ and R₇ form a cyclopropyl ring is a further preferred. Examples of compounds useful in the methods of the present invention include, but are not limited to those described in U.S. Patent Publication No. US 2004/0002461A1, such as 1-oxyl-4-(3′-ethoxy-2′,2′-dimethyl) propanecarbonyloxy-2,2,6,6-tetramethylpiperidine; 1-hydroxy-4-(3′-ethoxy-2′,2′-dimethyl) propanecarbonyloxy-2,2,6,6-tetramethylpiperidine hydrochloride; 1-oxyl-4-cyclopropanecarbonyloxy-2,2,6,6-tetramethylpiperidine; 1-hydroxy-4-cyclopropanecarbonyloxy-2,2,6,6-tetramethylpiperidine hydrochloride; 1-oxyl-4-(3′-benzyloxy-2′,2′-dimethyl) propanecarbonyloxy-2,2,6,6-tetramethylpiperidine; 1-hydroxy-4-(3′-benzyloxy-2′,2′-dimethyl) propanecarbonyloxy-2,2,6,6-tetramethylpiperidine hydrochloride; 1-hydroxy-4-(3′-hydroxy-2′,2′-dimethyl) propanecarbonyloxy-2,2,6,6-tetramethylpiperidine hydrochloride; 1-oxyl-4-(1-methyl-cyclopropane) carbonyloxy-2,2,6,6-tetramethylpiperidine; 1-hydroxy-4-(1-methyl-cyclopropane) carbonyloxy-2,2,6,6-tetramethylpiperidine hydrochloride; 1-oxyl-4-(2-furan) carbonyloxy-2,2,6,6-tetramethylpiperidine; 1-hydroxy-4-(2′-furan) carbonyloxy-2,2,6,6-tetramethylpiperidine hydrochloride; 1-oxyl-4-(3′-tetrahydrofuran) carbonyloxy-2,2,6,6-tetramethylpiperidine; 1-hydroxy-4-(3′-tetrahydrofuran) carbonyloxy-2,2,6,6-tetramethylpiperidine hydrochloride. 1-hydroxy-4-cyclopropanecarbonyloxy-2,2,6,6-tetramethylpiperidine hydrochloride, referred to herein as Compound 1, is particularly preferred.

While not wishing to be bound by theory, Applicants believe that Compound 1 (compound of formula 1, wherein R¹, R², R³, and R⁴ are methyl, R⁵ is H, and R⁶ and R⁷ taken together form a cyclopropane ring) and the other compounds of formula I are believed exert their anti-angiogenic and other therapeutic effects in two ways. First, the ester compounds are hydrolyzed in situ to form hydroxylamine components that exert therapeutic activity. Second, the esterified compounds themselves possess antioxidant activity, and therefore may possess complement activation inhibitory activity, thereby supporting the therapeutic efficacy of pharmaceutical preparations comprising the compounds.

In connection with the first basis for activity of the compounds of formula I, i.e., cleavage to liberate hydroxylamine components, numerous esterases are known to be present in various tissues and organs of the body. The specific esterase(s) that cleaves the esters of the present series need not be identified in order to practice the invention.

The compositions can be prepared in a wide variety of dosage forms according to any means suitable in the art for preparing a given dosage form. Pharmaceutically acceptable carriers can be either solid or liquid. Non-limiting examples of solid form preparations include powders, tablets, pills, capsules, lozenges, cachets, suppositories, dispersible granules, and the like. A solid carrier can include one or more substances which may also act as diluents, flavoring agents, buffering agents, binders, preservatives, tablet disintegrating agents, or an encapsulating material. Suitable solid carriers include magnesium carbonate, magnesium stearate, talc, sugar, lactose, pectin, dextrin, starch, gelatin, acacia, tragacanth, methylcellulose, sodium carboxymethyl-cellulose, polyethylene glycols, vegetable oils, agar, a low melting wax, cocoa butter, and the like. Non-limiting examples of suitable disintegrating agents include the cross-linked polyvinylpyrrolidone, agar, or alginic acid or a salt thereof such as sodium alginate. Non-limiting examples of liquid form preparations include solutions, suspensions, syrups, slurries, and emulsions. Suitable liquid carriers include any suitable organic or inorganic solvent, for example, water, alcohol, saline solution, physiological saline, buffered saline, dextrose solution, water propylene glycol solutions, and the like, preferably in sterile form.

The compositions can be formulated and administered to the subject as pharmaceutically acceptable salts. Non-limiting examples of pharmaceutically acceptable salts include acid addition salts such as those containing hydrochloride, sulfate, phosphate, sulfamate, acetate, citrate, lactate, tartrate, methanesulfonate, ethanesulfonate, benzenesulfonate, p-toluenesulfonate, cyclohexylsulfamate and quinate. Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid, according to means known and established in the art.

Aqueous solutions can be prepared by dissolving the active component in water and adding suitable colorants, flavors, stabilizing and thickening agents as desired. Aqueous suspensions can also be made by dispersing the finely divided active component in water with viscous material, such as natural or synthetic gums, resins, methylcellulose, sodium carboxymethylcellulose, and other well-known suspending agents.

Solid forms can be prepared according to any means suitable in the art. For example, capsules are prepared by mixing the composition with a suitable diluent and filling the proper amount of the mixture in capsules. Tablets are prepared by direct compression, by wet granulation, or by dry granulation. Their formulations usually incorporate diluents, binders, lubricants and disintegrators as well as the compound. Nonlimiting examples of diluents include various types of starch, cellulose, crystalline cellulose, microcrystalline cellulose, lactose, fructose, sucrose, mannitol, kaolin, calcium phosphate or sulfate, inorganic salts such as sodium chloride and powdered sugar. Powdered cellulose derivatives are also useful. Nonlimiting examples of tablet binders include starch, gelatin and sugars such as lactose, fructose, glucose and the like. Natural and synthetic gums can also be used, including acacia, alginates, methylcellulose, polyvinylpyrrolidine and the like. Polyethylene glycol, ethylcellulose and waxes can also serve as binders.

A lubricant can be used in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances which swell when-wetted to break up the tablet and release the compound, and include starches such as corn and potato starches, clays, celluloses, algins and gums, methylcellulose, agar, bentonite, wood cellulose, powdered natural sponge, cation-exchange resins, alginic acid, guar gum, citrus pulp, carboxymethyl cellulose, and sodium lauryl sulfate. Tablets can be coated with sugar as a flavor and sealant, or with film-forming protecting agents to modify the dissolution properties of the tablet. The compounds may also be formulated as chewable tablets, by using large amounts of pleasant-tasting substances such as mannitol in the formulation, as is now well-established in the art.

In some of the embodiments, the compositions can be administered orally. For such administrations, the pharmaceutical composition may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). These preparations may contain, in addition to the active agent, colorants, flavors, stabilizers, buffers, artificial and natural sweeteners, dispersants, thickeners, solubilizing agents, and the like. The compositions may be in powder form for constitution with a suitable vehicle such as sterile water, saline solution, or alcohol, before use.

Compositions for use in topical administration include, e.g., liquid or gel preparations suitable for penetration through the skin such as creams, liniments, lotions, ointments or pastes, and drops suitable for delivery to the eye, ear or nose.

In some embodiments, the present compositions include creams, drops, liniments, lotions, ointments and pastes are liquid or semi-solid compositions for external application. Such compositions may be prepared by mixing the active ingredient(s) in powdered form, alone or in solution or suspension in an aqueous or non-aqueous fluid with a greasy or non-greasy base. The base may comprise complex hydrocarbons such as glycerol, various forms of paraffin, beeswax; a mucilage; a mineral or edible oil or fatty acids; or a macrogel. Such compositions may additionally comprise suitable surface active agents such as surfactants, and suspending agents such as agar, vegetable gums, cellulose derivatives, and other ingredients such as preservatives, antioxidants, and the like.

The compositions can be formulated for application to the eye. Such compositions can be adapted for pharmaceutical use in intraocular injections, as an eye drop or in contact lenses, inserts or the like. Intraocular injections include without limitation intravitreous, intraretinal, and intramacula injections. Formulation of compounds into sterile water containing any desired diluents, salts, pH modifying materials and the like are known to persons skilled in the pharmaceutical formulations for producing formulations compatible with administration to the eye. It may be that injectables, eye drops, inserts, contact lenses, gels and other liquid forms may require somewhat different formulations. All such formulations consistent with direct administration to the eye are comprehended hereby.

A buffering agent may be used to maintain the pH of any ophthalmologic compositions of the invention, for example, eye drop formulations, in the range of about 4.0 to about 8.0; so as to minimize potential irritation to the eye. In certain embodiments, the pH is maintained at about 3.5 to about 6.0, preferably about 4.0 to about 5.5, in order to ensure that most of the hydroxylamine is in its protonated form for highest aqueous solubility. The buffer may be any weak acid and its conjugate base with a pKa of about 4.0 to about 5.5; e.g., acetic acid/sodium acetate; citric acid/sodium citrate. The pKa of the hydroxylamines is about 6.0. For direct intravitreal or intraocular injection, formulations should be at pH 7.2 to 7.5, preferably at pH 7.3-7.4.

Because the aqueous and vitreous humors exists in a highly reducing redox state, particularly nearest to the lens, it may be advantageous to include at least one reducing agent in ophthalmologic formulations in accordance with the invention, or to dose separately with a reducing agent to maintain the hydroxylamine in its reduced form. Preferred reducing agents may be N-acetylcysteine, ascorbic acid or a salt form, and sodium sulfite or metabisulfite, with ascorbic acid and/or N-acetylcysteine or glutathione being particularly suitable for injectable solutions. A combination of N-acetylcysteine and sodium ascorbate may be used in various formulations. A metal chelator antioxidant, such as EDTA (ethylenediaminetetraacetic acid) or possibly DTPA (diethylenetriaminepentaacetic acid) may also be added to keep the hydroxylamine in the reduced form.

The compositions can also be formulated for injection into the subject. For injection, the compositions of the invention can be formulated in aqueous solutions such as water or alcohol, or in physiologically compatible buffers such as Hanks's solution, Ringer's solution, or physiological saline buffer. The solution may contain formulatory agents such as suspending, stabilizing and/or dispersing agents. Injection formulations may also be prepared as solid form preparations which are intended to be converted, shortly before use, to liquid form preparations suitable for injection, for example, by constitution with a suitable vehicle, such as sterile water, saline solution, or alcohol, before use.

Some of the present compositions can be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions may further comprise one or more additional complement inhibitors. Such inhibitors can be any organic or inorganic compound, biomolecule, or fragment, analog, homolog, conjugate, or derivative thereof. Examples include soluble complement receptor 1 (sCR1), anti-C5 antibodies, CD59, and compstatin.

The compositions may further comprise one or more antioxidants. Exemplary reducing agents include mercaptopropionyl glycine, N-acetylcysteine, β-mercaptoethylamine, glutathione, ascorbic acid and its salts, sulfite, or sodium metabisulfite, or similar species. In addition. antioxidants can also include natural antioxidants such as vitamin E, C, leutein, xanthine, beta carotene and minerals such as zinc and selenium.

Techniques and formulations for administering above-described compositions may be found in Remington's Pharmaceutical Sciences, Meade Publishing Col., Easton, 20^{th} edition (2003).

Administration of the compositions can be by infusion or injection (intravenously, intraarterially, intramuscularly, intracutaneously, subcutaneously, intrathecal, intraduodenally, intraperitoneally, intraocularly, and the like). The compositions can also be administered intranasally, vaginally, rectally, orally, topically, or transdermally. Preferably, the compositions are administered orally. Administration can be at the direction of a physician.

For buccal administration, the compositions may take the form of tablets, troche or lozenge formulated in conventional manner. Compositions for oral or buccal administration, may be formulated to give controlled release of the active compound. Such formulations may include one or more sustained-release agents known in the art, such as glyceryl mono-stearate, glyceryl distearate and wax.

Compositions may be applied topically. Such administrations include applying the compositions externally to the epidermis, the mouth cavity, eye, ear and nose. This contrasts with systemic administration achieved by oral, intravenous, intraperitoneal and intramuscular delivery.

Various alternative pharmaceutical delivery systems may be employed. Non-limiting examples of such systems include liposomes and emulsions. Certain organic solvents such as dimethylsulfoxide also may be employed. Additionally, the compounds may be delivered using a sustained-release system, such as semipermeable matrices of solid polymers containing the therapeutic agent. The various sustained-release materials available are well known by those skilled in the art. Sustained-release capsules may, depending on their chemical nature, release the compounds over a range of several days to several weeks to several months.

The compositions can be topically delivered to the eye in eye drops or washes, or in a topical ophthalmic ointment. In some preferred embodiments, the compositions are delivered to the eye via periodic subconjunctival or intraocular injection. More preferred methods of administration will be those that provide for continuous administration to the eye, preferably into the macula and retina, and most preferably to areas of the eye where drusen can be deposited, such as proximal to the retina, or between the retinal pigmented epithelium and Bruch membrane.

The compositions may be applied in other ophthalmologic dosage forms known to those skilled in the art, such as pre-formed or in situ-formed gels or liposomes, for example as disclosed in U.S. Pat. No. 5,718,922 to Herrero-Vanrell. A direct injection of drugs into the vitreous cavity for treating other diseases of the eye has been described, in which microspheres or liposomes were used to release drugs slowly (Moritera, T. et al. “Microspheres of biodegradable polymers as a drug-delivery system in the vitreous” Invest. Ophthalmol. Vis. Sci. 1991 32(6):1785-90).

The composition can be delivered to or through the lens of an eye in need of treatment via a contact lens (e.g., Lidofilcon B, Bausch & Lomb CW79 or DELTACON (Deltafilcon A) or other object temporarily resident upon the surface of the eye. For example, U.S. Pat. No. 6,410,045 describes a contact lens-type drug delivery device comprising a polymeric hydrogel contact lens containing drug substance in a concentration of between 0.05% and 0.25% by weight absorbed in said contact lens which is capable of being delivered into the ocular fluid.

In other embodiments, supports such as a collagen corneal shield (e.g., BIO-COR dissolvable corneal shields, Summit Technology, Watertown, Mass.) can be employed. The compositions can also be administered by infusion into the eyeball, either through a cannula from an osmotic pump (ALZET®, Alza Corp., Palo Alto, Calif.) or by implantation of timed-release capsules (OCCUSENT®) or biodegradable disks (OCULEX®, OCUSERT®) which contain the compositions. These routes of administration have the advantage of providing a continuous supply of the composition to the eye. This may be an advantage for local delivery of the hydroxylamine compounds to vitreous humor.

Several other types of delivery systems are available that are particularly suitable for delivering pharmaceutical compositions to the interior of the eye. For instance, U.S. Pat. No. 6,154,671 to Parel et al. discloses a device for transferring a medicament into the eyeball by iontophoresis. The device utilizes a reservoir for holding the active agent, which contains at least one active surface electrode facing the eye tissue lying at the periphery of the cornea. The reservoir also has a return electrode in contact with the patient's partly closed eyelids. U.S. Pat. No. 5,869,079 to Wong et al. discloses combinations of hydrophilic and hydrophobic entities in a biodegradable sustained release ocular implant. In addition, U.S. Pat. No. 6,375,972 to Guo et al., U.S. Pat. No. 5,902,598 to Chen et al., U.S. Pat. No. 6,331,313 to Wong et al., U.S. Pat. No. 5,707,643 to Ogura et al., U.S. Pat. No. 5,466,233 to Weiner et al. and U.S. Pat. No. 6,251,090 to Avery et al. each describes intraocular implant devices and systems that may be used to deliver pharmaceutical compositions comprising compounds of the present invention.

Ocular implants for drug delivery to the eye are known in the art. For instance, U.S. Pat. No. 6,726,918 describes methods for treating inflammation-mediated conditions of the eye comprising: implanting into the vitreous of the eye of an individual a biodegradable implant comprising a steroidal anti-inflammatory agent and a biodegradable polymer, wherein the implant delivers the agent to the vitreous in an amount sufficient to reach a concentration equivalent to at least about 0.05 μg/ml dexamethasone within about 48 hours and maintains a concentration equivalent to at least about 0.03 μg/ml dexamethasone for at least about three weeks. Such implants are particularly suited for the methods of the present invention.

U.S. Pat. No. 6,713,081 describes ocular implant devices for the delivery of a therapeutic agent to an eye in a controlled and sustained manner. Dual mode and single mode drug delivery devices are illustrated and described. Implants suitable for subconjunctival and intravitreal placement are described. The patent also describes fabrication and implementation techniques associated with the ocular implant devices.

U.S. Pat. No. 6,429,194 describes aqueous ophthalmic preparations for instillation into the eye, or in which to pre soak or store an object to be inserted into the eye, such as a contact lens, an ointment, or a solid device to be inserted into the conjunctival sac. The ophthalmic preparation includes a mucin component, similar to that found at the normal human ocular surface.

U.S. Pat. No. 6,251,090 describes an intravitreal medicine delivery device, method and implant device through which a wide variety of beneficial medicines including drugs or other pharmacological agents can be introduced into the vitreous cavity over an extended period of time with only a single initial surgery to implant the device. The device and method minimize the surgical incision needed for implantation and avoid future or repeated invasive surgery or procedures. Additional amounts of the initial medicine can readily be introduced or the medication can be varied or changed, as required. Furthermore, the device and method allow the dosage delivered to the vitreous cavity to be controlled and allows the patient to control the timing of the delivery. The device is constructed so as to filter medicines delivered to the cavity and also avoids damage to or interference with other parts of the eye during implantation or during use.

U.S. Pat. No. 5,824,072 describes biocompatible ocular implants comprising active agents that are employed for introduction into a suprachoroidal space or an avascular region of an eye for therapeutic purposes. The administration of drugs is controlled and maintained for long periods of time, while ensuring the substantial absence of significant levels outside the site of administration.

U.S. Pat. No. 5,773,019 describes a continuous release drug delivery implant which, among other mentioned places, can be mounted either on the outer surface of the eye or within the eye. A drug core is covered by a polymer coating layer that is permeable to the low solubility agent without being release rate limiting.

U.S. Pat. No. 5,773,021 describes bioadhesive ophthalmic inserts that are placed in the conjunctival sac. The inserts are prepared by extrusion, thermoforming, or heat compression of a polymeric material matrix and the drug to be delivered. The polymeric matrix comprises a water-soluble biocompatible polymer, such as hydroxyalkyl celluloses, maltodextrins, chitosans, modified starches or polyvinyl alcohols; a water-insoluble biocompatible polymer such as an alkyl cellulose. Where applicable, a bioadhesive polymer such as polyvinyl carboxylic acid type polymers or certain bioadhesive polysaccharides or derivatives thereof may be used. The ophthalmic inserts are characterized therein as intended for the prolonged and controlled release of a medicinal substance.

U.S. Pat. Nos. 5,443,505 and 5,766,242 disclose implants comprising active agents for introduction into a suprachoroidal space or an avascular region of the eye, and describe placing microcapsules and plaques comprising hydrocortisone into the pars plana.

U.S. Pat. No. 5,378,475 describes a sustained-release implant for insertion into the vitreous of the eye. The implant has a first impermeable coating, such as ethylene vinyl acetate, surrounding most, but not all, of a drug reservoir and a second permeable coating, such as a permeable crosslinked polyvinyl alcohol, disposed over the first coating including the region where the first coating does not cover the drug reservoir, to provide a location through which the drug can diffuse out of the implant.

U.S. Pat. No. 5,725,493 describes an ocular implant device for providing drugs to the vitreous cavity over a period of time. The drug reservoir is attached to the outside of the eye with a passageway permitting medicament to enter the vitreous cavity of the eye.

U.S. Pat. No. 5,164,188 discloses encapsulated agents for introduction into the suprachoroid of the eye, and describes placing microcapsules and plaques comprising hydrocortisone into the pars plana.

U.S. Pat. No. 4,997,652 discloses biodegradable ocular implants comprising microencapsulated drugs, and describes implanting microcapsules comprising hydrocortisone succinate into the posterior segment of the eye.

U.S. Pat. No. 4,014,335 describes an ocular drug delivery device placed in the cul-de-sac between the sclera and lower eyelid for administering the drug and acting as a reservoir. The ocular device is characterized therein as administering drug to the eye in a controlled, continuous dosage rate over a prolonged time. To accomplish this, the ocular device comprises a three-layered laminate of polymeric materials holding the drug in a central reservoir region of the laminate. The drug diffuses from the reservoir through at least one of the polymeric layers of the laminate.

U.S. Pat. No. 4,300,557 teaches a capsule which can be filled with a pharmaceutical drug to be delivered which serves as an intraocular implant. The capsule is inserted in the vitreous region of the eye by making an incision in the eye, inserting the capsule and closing the incision. The capsule remains in place for a period of time and may be removed by making a second surgical incision into the eye and retrieving the device. The capsule has an attached tube which passes through the surface of the eye and extends outward from the eye useful for the subsequent injection of a drug. While in the vitreous, the device is not anchored and may move about freely.

Zhou et al. discloses a multiple-drug implant comprising 5-fluorouridine, triamcinolone, and human recombinant tissue plasminogen activator for intraocular management of proliferative vitreoretinopathy (PVR) (Zhou, T, et al. 1998, “Development of a multiple-drug delivery implant for intraocular management of proliferative vitreoretinopathy” J. Controlled Release 55:281-295).

The compositions utilized in accordance with the inventive methods may contain more than one hydroxylamine compound. In some embodiments, two or more hydroxylamines are administered simultaneously. In other embodiments, they are administered sequentially.

The compositions of the invention for treating complement-mediated or drusen-mediated pathologies may also be co-administered with other well known therapeutic agents that are selected for their particular usefulness against the condition that is being treated. For example, such therapeutic agents can be pain relievers, anti-inflammatory agents, antibiotics, anti-viral agents, anti-cirrhotic, or other known agents that treat or inhibit such pathologies.

The administration of these additional compounds may be simultaneous with the administration of the hydroxylamine compounds, or may be administered in tandem, either before or after the administration of the hydroxylamine compounds, as necessary. Any suitable protocol may be devised whereby the various compounds to be included in the combination treatment are administered within minutes, hours, days, or weeks of each other. Repeated administration in a cyclic protocol is also contemplated to be within the scope of the present invention.

To treat complement- or drusen-mediated pathologies in a subject, a therapeutically effective amount of a composition comprising a pharmaceutically acceptable carrier and at least one hydroxylamine compound or ester derivative thereof is administered to the subject. A therapeutically effective amount will provide a clinically significant decrease in localized or systemic inflammation, in the inhibition of drusen deposition, an inhibition or reduction in the rate of macula deterioration, and the like. The compositions can be administered to any animal, particularly mammals such as dogs, cats, rats, mice, rabbits, horses, pigs, cows, sheep, and donkeys, and are preferably administered to humans.

The effective amount of the composition may be dependent on any number of variables, including without limitation, the species, breed, size, height, weight, age, overall health of the subject, the type of formulation, the mode or manner or administration, or the severity of the complement- or drusen-mediated condition, or other related condition. The appropriate effective amount can be routinely determined by those of skill in the art using routine optimization techniques and the skilled and informed judgment of the practitioner and other factors evident to those skilled in the art. Preferably, a therapeutically effective dose of the compounds described herein will provide therapeutic benefit without causing substantial toxicity to the subject.

Toxicity and therapeutic efficacy of agents or compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents or compositions which exhibit large therapeutic indices are preferred. The dosage of such agents or compositions lies preferably within a range of circulating concentrations that include the ED50 with little or no toxicity. The dosage may vary within this range depending upon the dosage form employed and the route of administration utilized.

For the compositions used in the inventive methods, the therapeutically effective dose can be estimated initially from in vitro assays such as cell culture assays. For example, a dose can be formulated in animal models to achieve a circulating plasma concentration range that includes the IC50 as determined in cell culture (i.e., the concentration of the composition which achieves a half-maximal inhibition of the osteoclast formation or activation). Such information can be used to more accurately determine useful doses in a specified subject such as a human. The treating physician can terminate, interrupt, or adjust administration due to toxicity, or to organ dysfunctions, and can also adjust treatment as necessary if the clinical response was not adequate in order to improve the clinical response.

In the inventive methods, the compositions comprise a concentration of a hydroxylamine compound in a range of about 0.01% to about 90% of the dry matter weight of the composition. A daily dose range of about 0.01 mg/kg to about 100 mg/kg of the weight of the subject is preferred. Preferably, the daily dose ranges from about 0.1 mg/kg to about 50 mg/kg of the weight of the subject. More preferably, the daily dose ranges from about 1 mg/kg to about 10 mg/kg of the weight of the subject.

In preferred embodiments, administration of the compositions comprising hydroxylamine compounds to a subject will achieve a concentration of the hydroxylamine component in the range of about 0.1 μM to about 10 mM in the tissues and fluids of the subject, preferably in the eye, and more preferably in the macula or retina. In some embodiments, the range is from 1 μm to 5 mM, in other embodiments the range is about 10 μM to 2.5 mM. In still other embodiments, the range is about 50 μM to 1 mM. Most preferably the range of hydroxylamine concentration will be from 1 to 100 μM in the tissues and fluids of the subject, preferably in the eye, and more preferably in the macula or retina. In embodiments that include a reducing agent, either within the formulation or administered separately, the concentration of the reducing agent will be from 1 μM to 5 mM in the tissues and fluids of the subject to which the composition is administered, particularly in the eye, preferably in the range of 10 μM to 2 mM. The concentrations of the components of the composition are adjusted appropriately to the route of administration, by typical pharmacokinetic and dilution calculations, to achieve such local concentrations.

Treatment can be initiated with smaller dosages that are less than the optimum dose of the hydroxylamine compound, followed by an increase in dosage over the course of the treatment until the optimum effect under the circumstances is reached. If needed, the total daily dosage may be divided and administered in portions throughout the day.

For effective treatment of complement- or drusen-mediated conditions, one skilled in the art may recommend a dosage schedule and dosage amount adequate for the subject being treated. It may be preferred that dosing occur one to four times daily for as long as needed. The dosing may occur less frequently if the compositions are formulated in sustained delivery vehicles. The dosage schedule may also vary depending on the active drug concentration, which may depend on the needs of the subject.

The present invention is not limited to the embodiments described above, but is capable of variation and modification within the scope of the appended claims.

What is claimed:

1. A method for inhibiting complement activation in a subject, comprising administering to the subject a hydroxylamine compound or an ester derivative thereof in an amount effective to inhibit complement activation in the subject, wherein the ester derivative of hydroxylamine compound has the formula: 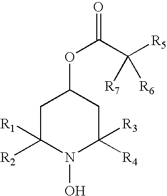
wherein:
R₁ and R₂ are, independently, H or C₁ to C₃alkyl;
R₃ and R₄ are, independently C₁ to C₃alkyl, or wherein R₁ and R₂, taken together, or R₃ and R₄, taken together, or R₁ and R₂, taken together and R₃ and R₄ taken together, are each cycloalkyl;
R₅ is H, OH, or C₁ to C₆alkyl;
R₆ is or C₁ to C₆alkyl, alkenyl, alkynyl, or substituted alkyl or alkenyl;
R₇ is C₁ to C₆alkyl, alkenyl, alkynyl, or substituted alkyl or alkenyl; or R₆ and R₇ taken together, or R₅, R₆, and R₇ taken together, form a carbocycle having from 3 to 7 atoms in the ring or form a heterocycle having from 3 to 7 atoms in the ring.

2. The method of claim 1, wherein the hydroxylamine compound has the structure:

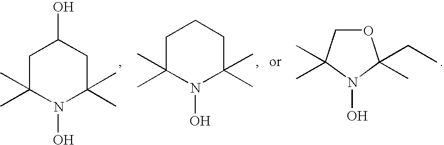

3. The method of claim 1, wherein R₁, R₂, R₃, and R₄ are each independently C₁-C₃alkyl.

4. The method of claim 1, wherein R₁, R₂, R₃, and R₄ are ethyl.

5. The method of claim 1, wherein R₁, R₂, R₃, and R₄ are methyl.

6. The method of claim 5, wherein R₅ is H or methyl, R₆ is methyl substituted with benzyloxy or C₁-C₆alkoxy, and R₇ is methyl.

7. The method of claim 5,wherein R₅ is H or methyl, and R₆ and R₇, taken together, form a cyclopropyl group.

8. The method of claim 5, wherein R₅, R₆, and R₇, taken together, form a furanyl group.

9. The method of claim 5, wherein R₅ is H, and R₆ and R₇, taken together, form a tetrahydrofuranyl group.

10. The method of claim 5, wherein R₅ is H, and R₆ and R₇, taken together, form a cyclopropyl group.

11. The method of claim 1, wherein the subject is a mammal.

12. The method of claim 11, wherein the mammal is a human.

13. The method of claim 1, wherein the hydroxylamine compound or ester derivative thereof inhibits the formation of C3a anaphylatoxin.

14. The method of claim 1, wherein the hydroxylamine compound or ester derivative thereof inhibits the formation of C5a anaphylatoxin.

15. A method for treating a subject having a pathology mediated by complement activation comprising administering to the subject a composition comprising a pharmaceutically acceptable carrier and at least one hydroxylamine compound or an ester derivative thereof in an amount effective to inhibit complement activation in the subject, wherein the ester derivative of the hydroxylamine compound has the formula: 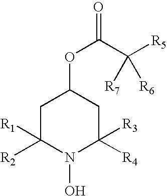
wherein:
R₁ and R₂ are, independently, H or C₁ to C₃alkyl;
R₃ and R₄ are, independently C₁ to C₃alkyl, or wherein R₁ and R₂, taken together, or R₃ and R₄, taken together, or R₁ and R₂, taken together and R₃ and R₄ taken together, are each cycloalkyl;
R₅ is H, OH, or C₁ to C₆alkyl;
R₆ is or C₁ to C₆alkyl, alkenyl, alkynyl, or substituted alkyl or alkenyl;
R₇ is C₁ to C₆alkyl, alkenyl, alkynyl, or substituted alkyl or alkenyl; or R₆ and R₇ taken together, or R₅, R₆, and R₇ taken together, form a carbocycle having from 3 to 7 atoms in the ring or form a heterocycle having from 3 to 7 atoms in the ring.

16. The method of claim 15, wherein the hydroxylamine compound has the structure:

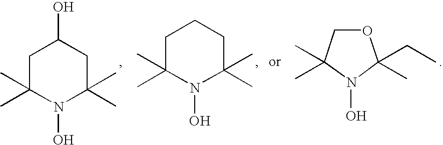

17. The method of claim 15, wherein R₁, R₂, R₃, and R₄ are each independently C₁-C₃alkyl.

18. The method of claim 15, wherein R₁, R₂, R₃, and R₄ are ethyl.

19. The method of claim 15, wherein R₁, R₂, R₃, and R₄ are methyl.

20. The method of claim 19, wherein R₅ is H or methyl, R₆ is methyl substituted with benzyloxy or C₁-C₆alkoxy, and R₇ is methyl.

21. The method of claim 19, wherein R₅ is H or methyl, and R₆ and R₇, taken together, form a cyclopropyl group.

22. The method of claim 19, wherein R₅, R₆, and R₇, taken together, form a furanyl group.

23. The method of claim 19, wherein R₅ is H, and R₆ and R₇, taken together, form a tetrahydrofuranyl group.

24. The method of claim 19, wherein R₅ is H, and R₆ and R₇, taken together, form a cyclopropyl group.

25. The method of claim 15, wherein the subject is a mammal.

26. The method of claim 25, wherein the mammal is a human.

27. The method of claim 15, wherein the composition is administered to the eye of the subject.

28. The method of claim 27, wherein the composition is administered to the macula of the eye.

29. The method of claim 27, wherein the composition is administered to the retina of the eye.

30. The method of claim 27, wherein the composition is administered to achieve in the eye of the subject a hydroxylamine concentration of about 0.1 μM to about 10 mM.

31. The method of claim 27, wherein the composition is administered to achieve in the eye of the subject a hydroxylamine concentration of about 1 μM to about 5 mM.

32. The method of claim 27, wherein the composition is administered to achieve in the eye of the subject a hydroxylamine concentration of about 10 μM to about 2.5 mM.

33. The method of claim 27, wherein the composition is administered to achieve in the eye of the subject a hydroxylamine concentration of about 50 μM to about 1 mM.

34. The method of claim 27, wherein the composition is administered to achieve in the eye of the subject a hydroxylamine concentration of about 1 μM to about 100 μM.

35. The method of claim 27, wherein the pathology is drusen formation.

36. The method of claim 27, wherein the pathology is macular degeneration.

37. The method of claim 36, wherein the macular degeneration is age-related macular degeneration.

38. The method of claim 15, wherein the hydroxylamine compound or ester derivative thereof inhibits the formation of C3a anaphylatoxin.

39. The method of claim 15, wherein the hydroxylamine compound or ester derivative thereof inhibits the formation of C5a anaphylatoxin.

40. A method to inhibit drusen formation in a subject comprising administering to the subject a hydroxylamine compound or ester derivative thereof in an amount effect to inhibit drusen formation in the subject, wherein the ester derivative of the hydroxylamine compound has the formula: 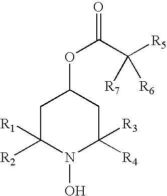
wherein:
R₁ and R₂ are, independently, H or C₁ to C₃alkyl;
R₃ and R₄ are, independently C₁ to C₃alkyl, or wherein R₁ and R₂, taken together, or R₃ and R₄, taken together, or R₁ and R₂, taken together and R₃ and R₄ taken together, are each cycloalkyl;
R₅ is H, OH, or C₁ to C₆alkyl;
R₆ is or C₁ to C₆alkyl, alkenyl, alkynyl, or substituted alkyl or alkenyl;
R₇ is C₁ to C₆alkyl, alkenyl, alkynyl, or substituted alkyl or alkenyl; or R₆ and R₇ taken together, or R₅, R₆, and R₇ taken together, form a carbocycle having from 3 to 7 atoms in the ring or form a heterocycle having from 3 to 7 atoms in the ring.

41. The method of claim 40, wherein the hydroxylamine compound has the structure:

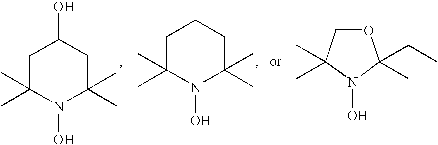

42. The method of claim 40, wherein R₁, R₂, R₃, and R₄ are each independently C₁-C₃alkyl.

43. The method of claim 40, wherein R₁, R₂, R₃, and R₄ are ethyl.

44. The method of claim 40, wherein R₁, R₂, R₃, and R₄ are methyl.

45. The method of claim 44, wherein R₅ is H or methyl, R₆ is methyl substituted with benzyloxy or C₁-C₆alkoxy, and R₇ is methyl.

46. The method of claim 44, wherein R₅ is H or methyl, and R₆ and R₇, taken together, form a cyclopropyl group.

47. The method of claim 44, wherein R₅, R₆, and R₇, taken together, form a furanyl group.

48. The method of claim 44, wherein R₅ is H, and R₆ and R₇, taken together, form a tetrahydrofuranyl group.

49. The method of claim 44, wherein R₅ is H, and R₆ and R₇, taken together, form a cyclopropyl group.

50. The method of claim 40, wherein the subject is a mammal.

51. The method of claim 40, wherein the mammal is a human.
